(19) United States
(12) Patent Application Publication
(10) Pub. No.: US 2024/0081678 A1
(43) Pub. Date: Mar. 14, 2024

(54) DEVICE FOR DETERMINING THE POSITION OF A PERSON'S PELVIS

(71) Applicant: SANLAS HOLDING GMBH, Graz (AT)

(72) Inventor: Cornelia ZENZMAIER, Laßnitzhöhe (AT)

(21) Appl. No.: 17/754,734

(22) PCT Filed: Sep. 24, 2020
(86) PCT No.: PCT/AT2020/060344

§ 371 Date: Apr. 11, 2022

(30) Foreign Application Priority Data
Oct. 16, 2019 (AT) A 50896/2019

(51) Int. Cl. A61B 5/103

(52) U.S. Cl. CPC A61B 5/1036; A61B 2562/0247

(57) ABSTRACT

A device for determining the location of a pelvis of a person, comprising at least one first pressure transmission element designed for absorbing a sitting pressure/contact pressure exerted by the coccyx/pubic bone of the person and at least one second pressure transmission element designed for absorbing a contact pressure exerted by the sacrum of the person and an evaluation unit, the evaluation unit being designed for performing the following steps: receiving measuring signals from the pressure transmission elements and evaluating them; determining a first characteristic pressure distribution in which, during a rotation of the person's pelvis around his or her horizontal axis, the first pressure transmission element is charged with a sitting pressure of the coccyx or the first pressure transmission element is charged with at least part of the contact pressure of the pubic bone; and determining a second characteristic pressure distribution in which the sitting pressure of the coccyx exerted on the first pressure transmission element exhibits a sudden drop and the contact pressure of the sacrum exerted on the second pressure transmission element exhibits a sudden rise.

The present invention relates to a device for determining the location of a person's pelvis, which is designed for accommodating the person, comprising at least one first pressure transmission element designed for absorbing a sitting pressure or, respectively, contact pressure exerted by the coccyx or, respectively, the pubic bone of the person and at least one second pressure transmission element designed for absorbing a contact pressure exerted by the person's sacrum and an evaluation unit connected to the pressure transmission elements.

The pelvis is considered to be the position controller for a neutral orientation of the functional spine and the lower extremities. Although the pelvis shows age- and gender-specific differences in terms of length and angular ratios, a universally valid, uniform description can still be given for its location-related stress. In seating systems, the person's pelvis is generally supported by the seating surface and the backrest of the seating system. The ischial tuberosities thereby serve for absorbing the weight of the upper body and can absorb a high sitting pressure when the person is in an ergonomically correct, essentially upright sitting position, whereas the coccyx and the sacrum remain essentially unstressed in this sitting position. In the horizontal dorsal position, the spine supports most of the weight force of the upper body and the body section weight in the area of the person's pelvis weighs on the sacrum, whereby the contact pressure exerted by the person's weight force is substantially at a maximum on the sacrum in this location and the coccyx remains essentially unstressed. In an ergonomically correct prone position, the person's pubic bone takes on the maximum portion of that body section weight.

People are often not aware of their own individual posture when standing, sitting and lying down and adopt positions that can cause one-sided painful muscle contractures and, in the long run, can lead to attrition processes, including herniated discs, in the event of incorrect loading of the spine and pelvis structure. In particular, sitting for long periods of time requires permanent static work from the muscles, often in a false posture and without sufficient compensatory movements, which causes muscular imbalances and promotes a wide range of physical complaints. By adopting such an ergonomically incorrect position, in particular an incorrect sitting position, the coccyx or sacrum can be loaded with parts of the person's weight force, which leads to a mismatch in the loads on the pelvis and the spinal structure, especially in the area of the transition of the pelvis into the spine, the sacroiliac joints. In addition, this may result in sprains and compressions throughout the spine, especially in the area of the lumbar spine, as the person is tempted to adopt a crooked posture due to substructures that are often soft and reduced muscle tension for maintaining an individually ergonomically correct sitting position in a passive sitting posture over an extended period of time, with the cause of this being found in the location-related position of the pelvis. This is particularly disadvantageous for people in need of care and/or people in wheelchairs, since they themselves cannot improve their incorrect sitting position due to their physical limitations, but have to rely on help from third parties, e.g., nursing staff. Since the above-mentioned persons often do not feel any pain, e.g., as a result of paraplegia or a neurodegenerative disease, or are unable to express pain, they are particularly at risk of sustaining permanent damage by adopting an incorrect sitting position.

In order to prevent this and to counteract possible incorrect loading of the spine and the associated risks of long-term physical damage, seating systems with seating surfaces which have a recess in the seating surface in the area of the coccyx are known from the prior art. Although these seating systems relieve the coccyx or, respectively, the region of the coccyx, they do not counteract an incorrect sitting position or, respectively, a rotation of the pelvis which is incorrect for the respective location, especially around the horizontal axis, since, for example, changes in the curvature of the spine, which may affect the location of the pelvis, are not detected. Other seating systems known from the prior art, e.g., modern car seats, use fluid-filled pressure elements that are inflatable by means of a pump device and comprise sensors in the area of the lumbar spine and/or in the area of the ischial tuberosities in order to achieve an ergonomically correct sitting position.

Such a car seat is disclosed in US20170086588A1, which comprises an air chamber system with two air chamber elements laterally spaced apart from one another and integrated into the seating surface of a seat, which, when sitting unevenly, positions the pelvis of the seated person by balancing his or her ischial tuberosities by supplying or discharging air by means of a compressor. In the air chamber elements, contact pressure sensors are incorporated which detect sitting pressures exerted on the seating surface by the user's ischial tuberosities. A controller evaluates the sitting pressure and controls the compressor according to the evaluation.

It is a disadvantage that, with this seating system, the respective individual location of the pelvis is not detected accurately, but only the sitting pressures transmitted from the ischial tuberosities to the air chamber elements are determined. Hence, this seating system is limited to horizontally balancing out the ischial tuberosities by rotating the pelvis around the sagittal axis. In case of an incorrect sitting position adopted by the person him- or herself, an over-inflated air chamber element can, for example, cause changes in the curvature of the spine, which may lead to persistent hyperextension of the lumbar spine due to the location-related incorrect rotation of the pelvis around the horizontal axis. By contrast, an under-inflated pressure element may lead to a hunchback with a causative posterior pelvic tilt, which entails the disadvantages that have already been mentioned above, in particular the strain on the coccyx or sacrum with a portion of the weight force of the person in an upright sitting position. This muscular overextension or compression of the spine with one-sided incorrect loading of the intervertebral discs due to a location-related incorrect pelvic position cannot be detected by the seating system of the above-mentioned prior art, which means that consequential physical damage cannot be prevented also in areas of the thoracic and cervical spine which are adjacent to the lumbar spine.

Furthermore, systems for monitoring the sitting posture of a person by means of pressure sensors incorporated in a chair or, respectively, by devices for measuring the contact pressures of a person lying down are known from the prior art (EP 3 011 896 A1, EP 1 093 755 A1). However, such systems or devices are not suitable for determining the location of a person's pelvis.

The applicant's earlier patent application A 50386/2019, which has not yet been published at the time of this patent application, discloses a device for positioning a person's body with control elements and sensors arranged on a seat element and on a back element. In this earlier patent application, the rolling process of the person during his or her transfer from an upright to a horizontal reclining position is mentioned, with the person's pelvis being positioned by linear adjusting movements via rotations about the sagittal, horizontal and longitudinal axes. A location of the pelvis is determined only indirectly by positioning the person's pelvis once a predetermined sitting and/or contact pressure difference is reached. As a result, the location of the pelvis cannot be determined exactly. In order to counteract an incorrect sitting position, it is therefore necessary to accurately determine the location of the person's pelvis, i.e., the inclination and rotation of the pelvis around the person's body axes, for every position of the person on a seating or reclining system.

It is the object of the present invention to provide a device for determining the location of a person's pelvis, which can be integrated into a pre-existing seating system or reclining system and which avoids the above-mentioned disadvantages of the prior art.

The present invention achieves the objects that have been posed by providing a device for determining the location of a person's pelvis having the features of claim 1.

In a further aspect, the invention provides a computer program product which comprises commands which, when the computer unit executes the program, prompt said unit to carry out the steps of claim 1.

In a further aspect, the invention provides a computer-readable data carrier on which the computer program product is stored.

Advantageous configurations of the invention become apparent from the dependent claims, the specification and the drawings.

The present invention provides a device, wherein the evaluation unit is designed for performing the following steps: receiving measuring signals from the pressure transmission elements and evaluating them, determining a first characteristic pressure distribution in which, during a rotation of the person's pelvis around his or her horizontal axis, the first pressure transmission element is charged for the first time with at least part of the sitting pressure of the coccyx or the first pressure transmission element is charged with at least part of the contact pressure of the pubic bone, and determining a second characteristic pressure distribution in which the sitting pressure of the coccyx exerted on the first pressure transmission element exhibits a sudden drop and the contact pressure of the sacrum exerted on the second pressure transmission element exhibits a sudden rise.

By determining the above-mentioned characteristic pressure distributions, the location of the pelvis in relation to the person's horizontal axis can be determined exactly. For this purpose, the person whose location of the pelvis is to be determined must be pre-positioned on the seating or reclining system, in case of persons in need of care by third parties, according to the arrangement of the pressure transmission elements on the seating system. After the characteristic pressure distribution has been determined, using means and methods known from the prior art, the person's pelvis can subsequently be positioned in such a way that a location-related ergonomically correct position of the pelvis can be maintained for a further possibility of a neutral orientation of the spine for every location on the seating system, which, in particular, is tilted around the person's horizontal axis. The above-mentioned physical complaints such as one-sided painful muscle contractures or signs of attrition on the spine can thus be reduced or even completely avoided, since the individually ergonomically correct orientation of the functional spine occurs in relation to the pelvis. In addition, the person sitting on a seating system can thus be positioned and supported on the seating system in accordance with his or her anatomy in a load-optimized way, wherein this neutral orientation of the functional spine can be maintained also in rotations about the sagittal or longitudinal axis.

The possibility of integrating the present invention into a pre-existing seating system or using it as a seat cover on a chair or bed results in a variety of possible applications, wherein the device is not limited to the following examples. The device could be integrated into the following entities, for example: into office chairs, wheelchairs, vehicle seats, child restraint systems, workout equipment, treatment beds, mattresses, and/or it could be used in the rehabilitation and therapy sector, in particular in standing beds, standing boards and/or operating beds. The integration of the present invention into operating beds makes sense particularly for procedures carried out in a prone or dorsal position, as they are often performed with inclined operating beds, as a result of which an undesirable relocation of the person located on the operating bed may happen, or, respectively, an incorrect positioning of the pelvis in relation to the orientation of the spine, in which the spine, especially in the lumbar region, is forced into a compressed or stretched position. Surgery on a pelvis positioned in this way can lead to a variety of problems. By means of the present invention, a position deviating from the neutral location-related position of the pelvis can be identified by detecting sitting and contact pressure changes between the first and the second pressure transmission element and this position can be counteracted by the above-mentioned means and methods. In this way, the neutral location-related position of the pelvis can be restored and maintained. Thus, in the course of an operation on operating beds with a mostly flat design, a procedure can be carried out with the best possible positioning of the body and possible surgical risks and, respectively, the sometimes very painful consequences of an incorrect positioning for hours on end can be minimized or even eliminated in particular by supporting the lumbar spine in a way that is matched to the position of the pelvis.

According to a preferred embodiment of the device for determining the location of a person's pelvis, the first pressure transmission element and the second pressure transmission element are arranged essentially at an obtuse angle to one another. In this way, the advantage is obtained that the first and the second characteristic pressure distribution can be determined also during a change in the inclination of the person's upper body, i.e., during a rolling process of his or her pelvis from a substantially upright sitting position to a substantially horizontal reclining position.

According to a preferred embodiment of the device for determining the location of a person's pelvis, the computer unit is furthermore designed for periodically evaluating the measuring signals transmitted by the pressure transmission elements to the computer unit at a predetermined time interval. As a result, the location of the pelvis can be monitored continuously, for example every second, and incorrect positioning of a person in need of care, for example by untrained staff, can be avoided.

According to a preferred embodiment of the device for determining the location of a person's pelvis, the evaluation unit is a membrane unit which comprises a membrane and is connected to the pressure transmission elements via channels and has a sensor arranged on the membrane and a computer unit which is designed for evaluating a sensor signal emitted by the sensor. The sitting and contact pressures exerted on the pressure transmission elements by the person's pelvis cause a redistribution in the pressure transmission elements of the fluid contained therein. This redistribution can be converted by the membrane unit into a sensor signal that can be evaluated by the computer unit, preferably an electrical sensor signal. As a result, the advantage is obtained that the device can respond quickly and reliably to a change in location or to a position of the pelvis that deviates from the neutral location-related position.

According to a further embodiment of the device for determining the location of a person's pelvis, the device comprises a third pressure transmission element and a fourth pressure transmission element, which each are arranged next to the second pressure transmission element and are designed for absorbing the contact pressures exerted by the person's iliac crests. In this way, it becomes possible to determine the location of the person's pelvis when it is rotated about the longitudinal axis, which is particularly advantageous for detecting micro- and macro-positioning in decubitus prophylaxis. With such acts of positioning, a shift in the centre of gravity of the body, at least in sections, is achieved, as a result of which certain areas of the body are relieved from pressure. A well-known form of positioning is the 30-degree inclined position, which, starting from an essentially horizontal dorsal position, is generally achieved with the help of two large pillows and burdens the right or left buttock or, respectively, half of the body. The device, which is integrated into a seating or reclining system of the type mentioned above, can detect the above-mentioned shift in the centre of gravity of the body section in the pelvic area and can determine the location of the pelvis in a lateral position and, with an additional rotation of the person around his or her longitudinal axis, also in a prone position of the person.

According to a further embodiment of the device for determining the location of a person's pelvis, the device comprises a fifth pressure transmission element and a sixth pressure transmission element, which each are arranged next to the first pressure transmission element and are designed for absorbing the sitting pressures exerted by the person's ischial tuberosities. The person's ischial tuberosities are spaced apart by a predetermined distance and define an ischial tuberosity level which, when the person has adopted an ergonomically correct sitting position, is horizontally balanced about the sagittal axis. For a variety of reasons, for example, as a result of sitting down for long periods or due to physical impairments, one of the two ischial tuberosities can be subjected to greater stress, which leads to a lopsided ischial tuberosity level and, in the long run, is harmful to the pelvic and spinal structure, especially the sacroiliac joints must be mentioned in this regard. By means of the additional fifth and sixth pressure transmission elements, the device can detect an asymmetrical sitting pressure load on the ischial tuberosities, and, by the computer unit forming a difference in the sitting pressures of the fifth and sixth pressure transmission elements, it can also determine the location of the pelvis in relation to the sagittal axis. The device can thereby determine the location of the pelvis with respect to all three body levels of the person, and an ergonomically correct load condition can thus be characterized in a precise and universally valid way for any positioning of the person, i.e., sitting and reclining positions, on a seating or reclining system.

This embodiment of the invention allows the device to determine not only the location of the pelvis in relation to the horizontal axis of the person, but also the location of the pelvis in relation to the sagittal and longitudinal axes. The information about the location of the pelvis can subsequently be used by a seating system as known from the prior art for correcting the above-mentioned asymmetrical sitting and contact pressure load or, respectively, for performing micro- or macro-positioning.

According to a further embodiment of the device for determining the location of a person's pelvis, the device comprises an array with a plurality of pressure transmission elements, which is designed for detecting the sitting and contact pressures of the person's pelvis in every position of the person on the device. As a result, the advantage is obtained that the above-mentioned step of pre-positioning the person on a seating system can be omitted, since, in this embodiment, not only the location but also the place of the pelvis on the seating system can be determined.

According to a preferred embodiment of the device for determining the location of a person's pelvis, the sensor is selected from the group consisting of mechanical, electrical, pneumatic or hydraulic sensors. It is thus possible to detect the sitting and/or contact pressures exerted by the person's pelvis using a plurality of sensors. For example, mechanical sensors such as strain gauges or also acceleration sensors can be used for detecting the sitting and/or contact pressures. If strain gauges are used, there may not be a requirement for a membrane unit, wherein the sensor signal emitted by the sensor can thereby be evaluated directly by the evaluation unit, e.g., a computer. The term “sensors” is not limited to the examples mentioned above.

According to a preferred embodiment of the device for determining the location of a person's pelvis, the device comprises a warning means which alerts the person when the first characteristic pressure distribution and/or the second characteristic pressure distribution has/have been determined. In this case, the warning means is selected from the group consisting of optical, acoustic or haptic warning means. In this way, it is rendered possible that persons that have adopted an ergonomically incorrect sitting or reclining position are made aware of their incorrect sitting or reclining position and thus develop more awareness of their posture. Regularly reminding someone of an incorrect sitting or reclining position can thus help to prevent physical damage of the type mentioned at the beginning. In case of people in need of care, the use of a warning means has the advantage that caregivers can be made aware of an ergonomically poor and/or incorrect positioning of the people in need of care, whereby the interval between checks performed by a caregiver can be extended.

The invention is now explained in further detail with reference to the drawings using non-limiting exemplary embodiments.

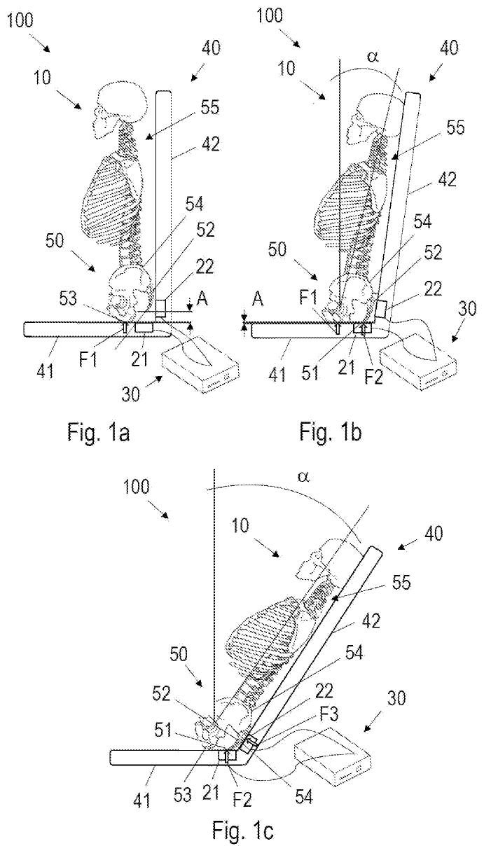

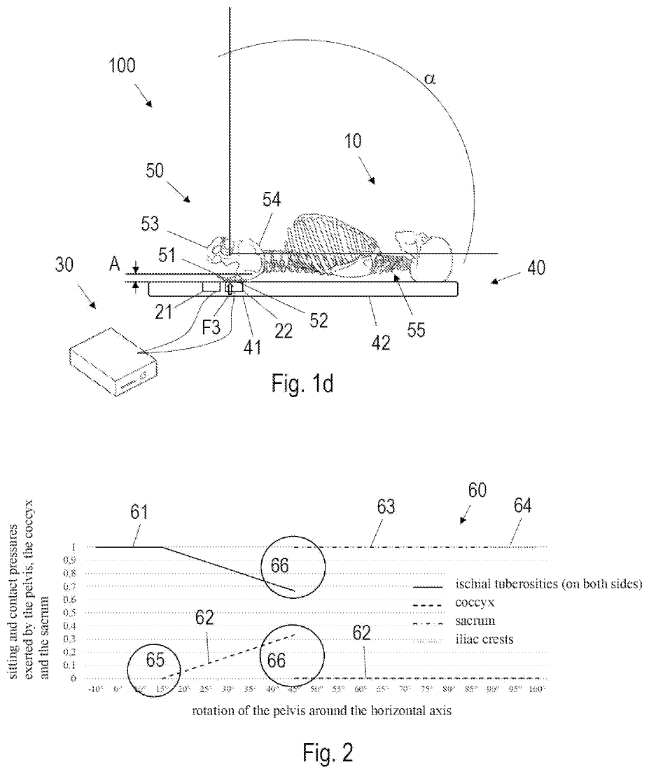

FIG. 1a shows a schematic illustration of the invention with a person located on an adjustable seating system in an ergonomically correct, upright sitting position.

FIG. 1b shows a schematic illustration of the invention with a person located on the adjustable seating system in an inclined position, with the person's coccyx exerting a sitting pressure on a first pressure element.

FIG. 1c shows a schematic illustration of the invention with a person located on the adjustable seating system in an inclined position, with the person's sacrum exerting a contact pressure on a second pressure element.

FIG. 1d shows a schematic illustration of the invention with a person located on the adjustable seating system in a horizontal reclining position, with the person's sacrum exerting a contact pressure on the second pressure element.

FIG. 2 shows a chart in which pressure profiles of the sitting and contact pressures exerted on the adjustable seating system during a rotation of the person's pelvis about the horizontal axis and markings which illustrate a first characteristic pressure distribution and a second characteristic pressure distribution according to claim 1 of the invention are depicted.

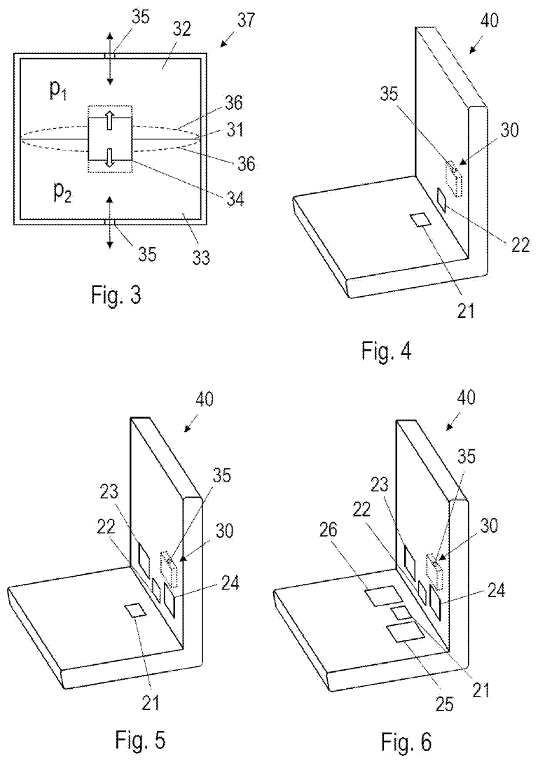

FIG. 3 shows a preferred embodiment of an evaluation unit.

FIG. 4 shows a perspective view of a seating system with pressure transmission elements arranged thereon and the evaluation unit of FIG. 3 according to a preferred embodiment of the invention.

FIG. 5 shows a perspective view of a seating system with pressure transmission elements arranged thereon and the evaluation unit of FIG. 3 according to a further embodiment of the invention.

FIG. 6 shows a perspective view of a seating system with pressure transmission elements arranged thereon and the evaluation unit of FIG. 3 according to a further embodiment of the invention.

Hereinafter, reference is made to FIGS. 1a to 1d, which show a person 10 in an upright, an inclined and a horizontally lying position on an adjustable seating system 40 with a seat element 41 and a back element 42, with the device 100 according to the invention being depicted as integrated therein. In this case, the device 100 can also be incorporated in a reclining system, which is not illustrated, or in a support, which is also not illustrated, and is not limited to the seating system 40 shown in FIGS. 1a to 1d. The device 100 is thus designed for accommodating the person 10 and comprises at least one first pressure transmission element 21 designed for absorbing a sitting pressure or, respectively, a contact pressure exerted by the coccyx 51 or, respectively, the pubic bone of the person 10, and at least one second pressure transmission element 22 designed for absorbing a contact pressure exerted by the person's sacrum 52. As shown in FIGS. 1b to 1d, the first pressure transmission element 21 and the second pressure transmission element 22 are arranged essentially at an obtuse angle to one another. The first pressure transmission element 21 is thereby arranged centrally in a rear third of the seat element 41, which faces the back element 42, and the second pressure transmission element 22 is also arranged centrally in a lower third of the back element 42, which faces the seat element 41. Fluid-filled, very flat chambers are preferably used as the pressure transmission elements 21, 22, the chambers preferably having a shape that is at least partially adapted to the pelvis 50 of the person 10. As a result, a higher sensitivity of the pressure transmission elements 21, 22 can be achieved, which allows sitting and contact pressure changes to be detected precisely. In particular, even slight shifts in the body's centre of gravity can be detected in this way. The term “fluid” is understood to mean any liquid or gas sufficient for the purpose of transmitting the sitting and contact pressures.

For example, water or air can be used as the fluid. The pressure transmission elements 21, 22 can also be simple strain gauges, e.g., metal foil strain gauges, which are subject to measurable changes in shape or, respectively, length, which depend on the magnitude of the sitting and contact pressures. For reasons of improved sitting and contact pressure detection, the pressure transmission elements 21, 22 are arranged visibly on the upper surface of the seat element 41, i.e., on the seating surface of the seat element 41, or respectively, on the upper surface of the back element 42, wherein the pressure transmission elements 21, 22 can be embedded in the seat element 41 and/or in the backrest 42 of the seating system 40 also in a non-visible manner.

FIGS. 1a to 1d show individual positions of the person 10 during his or her rotation about the horizontal axis, with FIG. 1a showing the person on a seating system 40 in an ergonomically correct upright sitting position and FIG. 1d showing the person 10 after the end of the rotation in an ergonomically correct horizontal reclining position. Arrows in FIGS. 1b-1d represent the sitting and contact pressures exerted by the pelvis 50 during said rotation, with F1 demonstrating the sitting pressure exerted by the ischial tuberosities, F2 demonstrating the sitting pressure exerted by the coccyx, and F3 demonstrating the contact pressure exerted by the sacrum.

As can be seen in FIG. 1a, in an ergonomically correct upright sitting position, the ischial tuberosities 53 are loaded with a large part of the weight force of the upper body of the person 10, wherein smaller portions of the weight force can be absorbed also by the lower extremities of the person, which are not illustrated. In this sitting position, the coccyx 51 is unstressed and is at a distance A from the seating surface of the seat element 41 of the seating system 40.

When the person 10 rotates through an angle α, FIG. 1b, the distance A is reduced, i.e., the distance from the coccyx 51 of the person 10 to the seating surface of the seating system 41 decreases and is essentially zero in this position. As a result, the coccyx 51 takes on a portion of the weight force and, in addition to the ischial tuberosities 53, exerts a sitting pressure on the seat element 41, which pressure is detected by the first pressure transmission element 21. This position corresponds to a tilt of the pelvis 50 of the person 10 by essentially 10° around the horizontal axis.

Starting from this position, the weight force components of the person 10 shift as said person rotates further in such a way that the sitting pressures exerted on the seat element 41 by the ischial tuberosities 53 are reduced or, respectively, become zero, and the coccyx 51 and the sacrum 52 absorb the bulk of the weight force. This position is illustrated in FIG. 1c and corresponds to a tilt of the pelvis 50 by essentially 45° around the horizontal axis of the person 10. In this case, the first pressure transmission element 21 detects the sitting pressure of the coccyx 51, and the second pressure transmission element 22 detects the contact pressure of the sacrum 52 of the person 10.

When the person 10 is transferred further into an essentially horizontal reclining position, FIG. 1d, the weight force components shift in such a way that, in this position, the sacrum 52 of the person 10 exerts a maximum contact pressure onto the second pressure transmission element 22. As can be seen from the distance A, the coccyx 51 does not exert any contact pressure onto the first pressure transmission element 21 in this position.

In addition to the pressure transmission elements 21, 22 that have already been mentioned, the device 100 according to the invention comprises an evaluation unit 30 which is connected to the pressure transmission elements 21, 22 preferably via channels, with the evaluation unit 30 being designed for receiving measuring signals from the pressure transmission elements 21, 22 and evaluating them. The measuring signal is a physical variable such as, for example, pressure, electrical current or electrical voltage and depends on the type of pressure transmission elements 21, 22 used. In the preferred embodiment mentioned above, in which fluid-filled chambers are used as pressure transmission elements 21, 22, the measuring signal is a pressure signal, which is transmitted to the evaluation unit 30 via the channels.

FIG. 2 shows a chart 60 with series of measurements 61, 62, 63, 64, which illustrates pressure profiles of the sitting and contact pressures exerted on said seating system 40 during the above-described rotation of the pelvis 50 of the person 10. The scale of the ordinate of the chart 60 is depicted as normalized, with 0 indicating no sitting or, respectively, contact pressure and 1 indicating a maximum sitting or, respectively, contact pressure. The series of measurements 61 shows the pressure profile of the sitting pressures exerted by the ischial tuberosities 53, with the series of measurements 62 showing the pressure profile of the sitting pressure exerted by the coccyx 51, the series of measurements 63 showing the pressure profile of the contact pressure exerted by the sacrum and the series of measurements 64 showing the pressure profile of the contact pressures exerted by the iliac crests 54.

Furthermore, the evaluation unit 30 is designed for determining a first characteristic pressure distribution 65 in which, during a rotation of the pelvis 50 of the person 10 around its horizontal axis, the first pressure transmission element 21 is charged for the first time with at least part of the sitting pressure of the coccyx 51 or the first pressure transmission element 21 is charged with at least part of the contact pressure of the pubic bone. The first characteristic pressure distribution 65, in which the coccyx 51 of the person 10 is charged for the first time with at least part of the sitting pressure, is shown in FIG. 2 with an inclination of the pelvis 50 by 15° around the horizontal axis. Depending on the flexibility of a substructure, for example, the seating surface of the seat element 41 of the seating system 40 and/or due to a support of the spine 55 on the back element 42 and/or pathological changes in the coccyx 51, the coccyx 51 can be charged with a sitting pressure already at an inclination of the pelvis 50 of essentially 10° and more around the horizontal axis. In the prone position, the pubic bone of the person 10 exerts a contact pressure onto the first pressure transmission element 21 in accordance with the contact pressure of the sacrum 52 of the person 10 in the dorsal position, as shown in the series of measurements 63 in FIG. 2.

Furthermore, the evaluation unit 30 is designed for determining a second characteristic pressure distribution 66 in which the sitting pressure of the coccyx 51 exerted on the first pressure transmission element 21 exhibits a sudden drop and the contact pressure of the sacrum 52 exerted on the second pressure transmission element 22 exhibits a sudden rise. This second characteristic pressure distribution 66 is illustrated in FIG. 2.

The evaluation and reception of the measuring signals from the pressure transmission elements 21, 22 as well as the determination of the first characteristic pressure distribution 65 and the second characteristic pressure distribution 66 is implemented by a computer program product which comprises commands which, when the evaluation unit 30 executes the program, prompt said unit to carry out the above-mentioned steps. In this case, the computer program product is stored on a computer-readable data carrier.

The characteristic pressure distributions 65, 66 are preferably determined by the evaluation unit 30 by determining pressure differences of the sitting and contact pressures exerted on the pressure transmission elements 21, 22 by the pelvis 50, the coccyx 51 and the sacrum 52 of the person 10. However, the characteristic pressure distributions 65, 66 can also be determined via pattern recognition by comparing the sitting and contact pressures with a predetermined pressure pattern.

FIG. 3 shows a preferred embodiment of the evaluation unit 30. Accordingly, the evaluation unit 30 is a membrane unit 37 which comprises a membrane 31 and is connected to the pressure transmission elements 21, 22 via channels, which are not illustrated, and preferably evaluates the measuring signals transmitted from the pressure transmission elements 21, 22 to the evaluation unit 30 periodically at a predetermined time interval. Thus, the measuring signals can be evaluated, for example, every second in order to be able to reliably identify the location of the pelvis 50 of the person 10. The membrane 31 shows a linearly elastic behaviour and divides the membrane unit 37 into two chambers 32, 33, with a first chamber 32 having a pressure p₁ and a second chamber 33 having a pressure p₂. In addition, the chambers 32, 33 each have an opening 35 at which the channels are arranged. Furthermore, the membrane unit 37 comprises a sensor 34 arranged on the membrane 31 and a computer unit, which is not illustrated. The sensor 34 is preferably arranged centrally on the membrane 31. The sitting and contact pressures exerted on the seat element 41 and the back element 42 of the seating system 40 by the coccyx 51 and the sacrum 52 of the person 10 cause a redistribution of the fluid contained in the pressure transmission elements 21, 22, whereby a pressure difference is generated in the chambers 32, 33, which leads to a flat membrane deflection 36. The sensor 34 arranged on the membrane 31 is preferably a sensor selected from the group consisting of mechanical, electrical, pneumatic or hydraulic sensors, but in particular an acceleration sensor. By detecting a change in speed, the acceleration sensor can detect the membrane deflection 36 and transmit a corresponding sensor signal to the computer unit. Subsequently, the computer unit evaluates the sensor signal, with the sensor signal preferably being in direct relation to the membrane deflection 36. If the first pressure transmission element 21 is connected to the first chamber 32 and the second pressure transmission element 22 is connected to the second chamber 33 of the membrane unit 37, a pressure level of p₁>p₂ prevails in the first characteristic pressure distribution 65. If the sacrum 52 of the person 10 exerts a greater contact pressure in comparison to the sitting pressure exerted by the coccyx 51, as is the case with the second characteristic pressure distribution 66, a pressure level of p₂>p₁ is reached. If the coccyx 51 exerts a sitting pressure on the first pressure transmission element 21 which is as high as a contact pressure exerted on the second pressure transmission element 22 by the sacrum 52, p₁=p₂ applies and no membrane deflection occurs. This also corresponds to a load-free state of the pressure transmission elements 21, 22, i.e., the pressure transmission elements 21, 22 are not charged with a sitting and/or contact pressure. The acceleration sensor is preferably incorporated in a computer unit, which is not illustrated. For example, a processor can be arranged on the membrane 31, which has a corresponding acceleration sensor and evaluates the sensor signals of the acceleration sensor.

FIG. 4 schematically shows the preferred embodiment of the present invention in a perspective view. The membrane unit 37 is preferably arranged in the area of the pressure transmission elements 21, 22, and preferably it is integrated into the back element 42 of the seating system 40 in a non-visible manner. However, the membrane unit 37 is also usable while being detached from the seating system 40, in which case the length of the channels, which connect the membrane unit 37 to the pressure transmission elements 21, 22 and are not shown in this figure, can be significantly increased. The flat deflection 36 of the sensor 34 in the membrane unit 37, which sensor is herein arranged by way of example, occurs in this case in the frontal plane in a longitudinal direction.

In a further embodiment, the device 100 according to the invention can comprise a third pressure transmission element 23 and a fourth pressure transmission element 24, as illustrated in FIG. 5, which each are arranged next to the second pressure transmission element 22 and are designed for absorbing the contact pressures exerted by the iliac crests 54 of the person 10. These additional pressure transmission elements 23, 24 essentially have the same shape as the first pressure transmission element 21 and the second pressure transmission element 22 and are preferably also designed as fluid-filled chambers. Due to the third pressure transmission element 23 and the fourth pressure transmission element 24, a membrane deflection can take place also in the horizontal direction, in addition to that in the longitudinal direction. In this case, a membrane deflection in the horizontal direction corresponds to a rotation of the pelvis 50 around the longitudinal axis of the person 10. For this purpose, the membrane unit 37 comprises a second membrane, which is not shown in the figures and which is arranged in such a way that it divides the membrane unit 37 into two additional chambers, which are also not shown, i.e., a total of four chambers. In this case, the acceleration sensor is preferably arranged at a crossing point of the first membrane and the second membrane and detects the flat membrane deflection both in the longitudinal and in the horizontal direction.

In a further embodiment of the invention, which is illustrated in FIG. 6, the device 100 comprises a fifth pressure transmission element 25 and a sixth pressure transmission element 26, which each are arranged next to the first pressure transmission element 21 and are designed for absorbing the contact pressures exerted by the ischial tuberosities 53 of the person 10. The fifth pressure transmission element 25 and the sixth pressure transmission element 26 have essentially the same shape as the first pressure transmission element 21 and the second pressure transmission element 22 and are preferably also designed as fluid-filled chambers. In this embodiment, the membrane unit 37 is designed in such a way that a membrane deflection occurs both in the longitudinal and in the horizontal direction. During the act of sitting down, the membrane is subject to a maximum membrane deflection due to the maximum sitting pressures exerted on the fifth pressure transmission element 25 and the sixth pressure transmission element 26 by the ischial tuberosities 53, as illustrated in FIG. 2. In this way, the weight force of the upper body of the person 10 can be determined.

In this embodiment and the above-mentioned embodiment of the invention, namely that with four pressure transmission elements 21, 22, 23, 24, the location of the pelvis 50 of the person 10 can be detected also in case of mixed movements. Mixed movements can, for example, be movements of the person 10 which take place simultaneously around the horizontal axis and the sagittal axis. This movement can occur, for example, if the ischial tuberosities 53 are stressed asymmetrically and a rotation of the pelvis 50 of the person 10 around his or her horizontal axis happens at the same time. In this case, the membrane deflection takes place both in the longitudinal and in the horizontal direction. A membrane deflection in the sagittal direction caused by the sitting and contact pressures of the pelvis 50 is blocked by the above-described structure of the membrane unit 37. Changes in the sensor signal in this plane can be attributed to changes in the position of the seat element 41 of the seating system 40, whereby it is also possible to deduce the corresponding change in the position of the back element 42. Rotations of the seating system 40 around the longitudinal axis, which corresponds to a sideways inclination of the seat element 41, and around the horizontal axis, which corresponds to an upward or downward inclination of the seat element 41, can thereby be determined. A resulting maximum sitting and/or contact pressure can be calculated from this by the evaluation unit 30 via a triangle of forces calculation. From this, the device can determine sitting and/or contact pressure components of the pelvis, whereby the location of the pelvis 50 can be determined even if it is in a slightly defective position.

In an embodiment of the invention which is not shown in the figures, the device 100 comprises an array with a plurality of pressure transmission elements, which is designed for detecting the sitting and contact pressures of the pelvis 50 of the person 10 in every position of the person 10 on the device 100. The array with a plurality of pressure transmission elements can be incorporated in the seat element 41, in the back element 42 or in both, i.e., in the seat element 41 and in the back element 42. As a result, the pre-positioning of the person 10 on the seating system 40 can be omitted.

In an embodiment which is not shown in the figures, the device 100 comprises a warning means which alerts the person 10 when the first characteristic pressure distribution 65 and/or the second characteristic pressure distribution 66 has/have been determined. If the first characteristic pressure distribution 65 and/or the second characteristic pressure distribution 66, i.e., an ergonomically incorrect sitting or reclining position, have/has been determined by the evaluation unit 30, acoustic signals in the form of a sound emitted by a loudspeaker or optical signals in the form of a warning light emitted by a warning lamp can alert the person 10, for example. In this case, the warning means can be incorporated/arranged in/on the seating system 40 or can be located in the vicinity of the seating system 40 while being detached from it. In this case, the warning means are selected from the group consisting of optical, acoustic or haptic warning means.

According to the above-described exemplary embodiment with surface sensors, a rotation of the pelvis about the horizontal axis essentially by 45° creates the angle that is essential to the invention, at which the weight force components of the person on the seat element pass from the coccyx and the ischial tuberosities to the sacrum. However, this angle, which is essential to the invention, also depends on the weight of the respective person and the hardness of the seat element. Accordingly, this angle, which is essential to the invention, can be in an angular range beginning at 110, which is where the coccyx starts to press on the seat element, and ending at 56°, which is where the ischial tuberosities remove the load from the seat element. Therefore, the angle, which is essential to the invention, can be, for example, 35°, 40°, 43°, 45°, 47°, 50°, 530 or 56°.

According to a further exemplary embodiment with a particularly soft seat cover, the angular range described above for surface sensors and having an angle essential to the invention of at least 110 does not constitute the relevant limit. With a particularly soft seat cover, the pressure of the coccyx can actually already be measured in an upright sitting position, i.e., at an angle essential to the invention of 0°, and the pressure of the ischial tuberosities can be measured in a reclining position, i.e., at an angle essential to the invention of 90°, which is why, for particularly soft seat covers, the angular range of the angle essential to the invention should be set to 0° to 90°.

1.-11. (canceled)

12. A device for determining the location of the pelvis of a person, which device is designed for accommodating the person, comprising at least one first pressure transmission element designed for absorbing a sitting pressure or, respectively, contact pressure exerted by the coccyx or, respectively, the pubic bone of the person and at least one second pressure transmission element designed for absorbing a contact pressure exerted by the sacrum of the person and an evaluation unit connected to the pressure transmission elements, characterized in that the evaluation unit is designed for performing the following steps:
a) receiving measuring signals from the pressure transmission elements and evaluating them;
b) determining a first characteristic pressure distribution in which, during a rotation of the pelvis of the person around his or her horizontal axis, the first pressure transmission element is charged for the first time with at least part of the sitting pressure of the coccyx or the first pressure transmission element is charged with at least part of the contact pressure of the pubic bone; and
c) determining a second characteristic pressure distribution in which the sitting pressure of the coccyx exerted on the first pressure transmission element exhibits a sudden drop and the contact pressure of the sacrum exerted on the second pressure transmission element exhibits a sudden rise.

13. A device according to claim 12, characterized in that the first pressure transmission element and the second pressure transmission element are arranged essentially at an obtuse angle to one another.

14. A device according to claim 12, characterized in that the evaluation unit is furthermore designed for periodically evaluating the measuring signals transmitted by the pressure transmission elements to the evaluation unit at a predetermined time interval.

15. A device according to claim 12, characterized in that the evaluation unit is a membrane unit which comprises a membrane and is connected to the pressure transmission elements via channels and has a sensor arranged on the membrane and a computer unit which is designed for evaluating a sensor signal emitted by the sensor.

16. A device according to claim 12, characterized in that the device comprises a third pressure transmission element and a fourth pressure transmission element, which each are arranged next to the second pressure transmission element and are designed for absorbing the contact pressures exerted by the iliac crests of the person.

17. A device according to claim 16, characterized in that the device comprises a fifth pressure transmission element and a sixth pressure transmission element, which each are arranged next to the first pressure transmission element and are designed for absorbing the sitting pressures exerted by the person's ischial tuberosities.

18. A device according to claim 12, characterized in that the device comprises an array with a plurality of pressure transmission elements, which is designed for detecting the sitting and contact pressures of the pelvis of the person in every position of the person on the device.

19. A device according to claim 15, characterized in that the sensor is selected from the group consisting of mechanical, electrical, pneumatic or hydraulic sensors.

20. A device according to claim 12, comprising a warning means, characterized in that the warning means alerts the person when the first characteristic pressure distribution and/or the second characteristic pressure distribution has/have been determined, with the warning means being selected from the group consisting of optical, acoustic or haptic warning means.

21. A computer program product, comprising commands which, when the evaluation unit executes the program, prompt said unit to carry out the steps of claim 12.

22. A computer-readable data carrier on which the computer program product according to claim 21 is stored.
